(19) United States
(12) Patent Application Publication
(10) Pub. No.: US 2022/0296147 A1
(43) Pub. Date: Sep. 22, 2022

(54) ELECTRODE FOR ORGANISM

(71) Applicant: Nippon Telegraph and Telephone Corporation, Tokyo (JP)

(72) Inventors: Hiroto Matsuoka, Tokyo (JP); Takako Ishihara, Tokyo (JP); Hiroyoshi Togo, Tokyo (JP); Kazuyoshi Ono, Tokyo (JP)

(21) Appl. No.: 17/636,489

(22) PCT Filed: Sep. 3, 2019
(86) PCT No.: PCT/JP2019/034575

§ 371 Date: Feb. 18, 2022

(51) Int. Cl. A61B 5/268

(52) U.S. Cl. CPC A61B 5/268; A61B 2562/125; A61B 2562/164

(57) ABSTRACT

A bioelectrode includes an annular sheet that is annular in a plan view, an annular metal-made wiring formed on the annular sheet, a conductive sheet formed on the wiring, a connection wiring, and an adhesive layer formed so as to cover the conductive sheet. The bioelectrode can be attached, via the adhesive layer, onto the back surface of a garment. The annular sheet is configured by a material with insulation, waterproofness, and flexibility and has an opening at a central part thereof. The conductive sheet is formed on and in contact with the wiring, and is electrically connected to the wiring. Further, the conductive sheet is formed on the annular sheet so as to cover the wiring and close the opening.

### CROSS-REFERENCE TO RELATED APPLICATIONS

This application is a national phase entry of PCT Application No. PCT/JP2019/034575, filed on Sep. 3, 2019, which application is hereby incorporated herein by reference.

### TECHNICAL FIELD

The present invention relates to a bioelectrode.

### BACKGROUND

Measuring the electrocardiogram or cardiac rate is a technique useful in a wide range of fields, including not only the diagnosis of heart diseases but also physical condition management such as prevention of heat stroke, judgement of central fatigue, and detection of drowsiness, as well as sports such as cardiac beat training. For example, in order to easily measure the electrocardiogram or cardiac beat, there are garments capable of measuring the electrocardiogram or cardiac beat when they are worn, including “hitoe (registered trademark)”.

This kind of functional garment includes, for example, as illustrated in FIG. 7, two bioelectrodes 200 provided on the back of a shirt 251 for detection of the electrocardiogram. The bioelectrode 200 is a conductive fabric using fibers coated with conductive polymers. The bioelectrode 200 made of this conductive fabric is sewed onto the back surface of the shirt 241. Further, a measuring device 211 attached to this shirt measures the electrocardiogram. The measuring device 211 can measure the electrocardiogram from the electric potential difference occurring at the time of muscle contraction of the heart, measured by the two bioelectrodes 200. Further, the measuring device 211 has a wireless communication function to transmit the measured electrocardiogram or cardiac rate to a terminal device such as a smartphone (Non-Patent Literature 1).

When measuring the electrocardiogram, a doctor or a laboratory technician will attach the electrode to a predetermined location for the measurement. Using the above described garment can bring the electrode into a state where the electrode is located and attached at an appropriate position, just when the garment is worn. Therefore, a user can easily measure the electrocardiogram or cardiac beat, and can easily receive services utilizing measurement results.

The above described technique has a problem that, when the shirt 251 gets wet with sweat or the like, the resistance between the two bioelectrodes 200 attached to the shirt 251 decreases and the measurable cardiac potential is lowered. As a method for solving this problem, a technique for attaching the bioelectrodes 200 to the back surface of the shirt 251 via insulating tapes 201 has been proposed (Patent Literature 1), as illustrated in FIG. 8.

### CITATION LIST

### Patent Literature

Patent Literature 1: International Publication No. 2016/093194

### Non-Patent Literature

Non-Patent Literature 1: Shingo Tsukada et al., “Wearable electrode inner that measures the electrocardiogram just by wearing”, NTT Technical Journal, vol. 26, no. 2, pp. 15-18, 2014.

### SUMMARY

### Technical Problem

However, the above described technique has a problem that the conductivity of the bioelectrode using conductive polymers is not so high.

Embodiments of the present invention have been made to solve the above problem and intends to increase the conductivity of the bioelectrode using conductive polymers.

### Means for Solving the Problem

A bioelectrode according to embodiments of the present invention includes an annular sheet with insulation, waterproofness, and flexibility, which is annular and has an opening at a central part thereof, an annular metal-made wiring formed on the annular sheet, a conductive sheet formed on the annular sheet so as to cover the wiring and close the opening and configured by conductive polymers, a connection wiring connected to the wiring and drawn out from an annular part of the annular sheet to the outside, and an adhesive layer formed on the conductive sheet so as to cover the conductive sheet.

### Effects of Embodiments of the Invention

As described above, according to embodiments of the present invention, the annular metal-made wiring is provided on the annular sheet that is annular and has the opening at the central part thereof, and the conductive sheet is formed on the annular sheet so as to cover the wiring and close the opening. Therefore, the conductivity of the bioelectrode using conductive polymers can be increased.

### BRIEF DESCRIPTION OF THE DRAWINGS

FIG. 1A is a plan view illustrating a configuration of a bioelectrode 100 according to an embodiment of the present invention.

FIG. 1B is a plan view illustrating the configuration of the bioelectrode 100 according to the embodiment of the present invention.

FIG. 1C is a cross-sectional view illustrating a partial configuration of the bioelectrode 100 according to the embodiment of the present invention.

FIG. 1D is a cross-sectional view illustrating a partial configuration of the bioelectrode 100 according to the embodiment of the present invention.

FIG. 2A is a plan view illustrating a state of the bioelectrode in a manufacturing process according to an embodiment of the present invention.

FIG. 2B is a plan view illustrating a state of the bioelectrode in a manufacturing process according to the embodiment of the present invention.

FIG. 2C is a plan view illustrating a state of the bioelectrode in a manufacturing process according to the embodiment of the present invention.

FIG. 2D is a plan view illustrating a state of the bioelectrode 100 in a manufacturing process according to the embodiment of the present invention.

FIG. 2E is a plan view illustrating a configuration of the bioelectrode 100 according to the embodiment of the present invention.

FIG. 3 is a configuration diagram illustrating an application example of the bioelectrode 100 according to the embodiment of the present invention.

FIG. 4 is a plan view illustrating a state of a bioelectrode 100a in a manufacturing process according to an embodiment of the present invention.

FIG. 5A is a plan view illustrating a detailed configuration of bioelectrodes.

FIG. 5B is a bottom view illustrating a detailed configuration of the bioelectrodes.

FIG. 5C is a right side view illustrating a detailed configuration of the bioelectrodes.

FIG. 5D is a left side view illustrating a detailed configuration of the bioelectrodes.

FIG. 5E is a front view illustrating a detailed configuration of the bioelectrodes.

FIG. 5F is a rear view illustrating a detailed configuration of the bioelectrodes.

FIG. 6A is a plan view illustrating a detailed configuration of a bioelectrodes according to an embodiment of the present invention.

FIG. 6B is a bottom view illustrating a detailed configuration of the bioelectrodes according to the embodiment of the present invention.

FIG. 6C is a right side view illustrating a detailed configuration of the bioelectrodes according to the embodiment of the present invention.

FIG. 6D is a left side view illustrating a detailed configuration of the bioelectrodes according to the embodiment of the present invention.

FIG. 6E is a front view illustrating a detailed configuration of the bioelectrodes according to the embodiment of the present invention.

FIG. 6F is a rear view illustrating a detailed configuration of the bioelectrodes according to the embodiment of the present invention.

FIG. 6G is a partial cross-sectional view illustrating a detailed configuration of the bioelectrodes according to the embodiment of the present invention.

FIG. 7 is a plan view illustrating a configuration of electrocardiography using a bioelectrodes 200.

FIG. 8 is a plan view illustrating a configuration of electrocardiography using the bioelectrodes 200.

### DETAILED DESCRIPTION OF ILLUSTRATIVE EMBODIMENTS

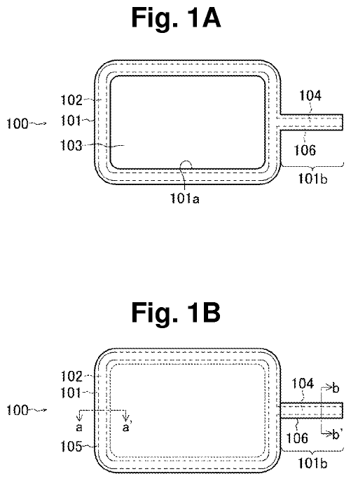

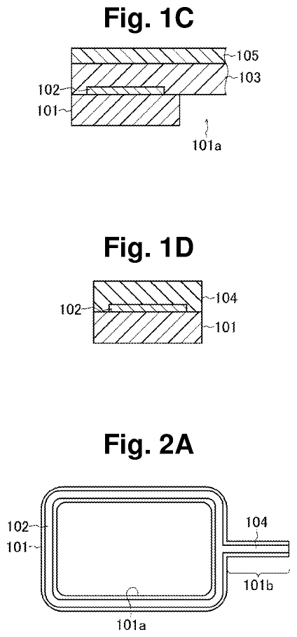

Hereinafter, a bioelectrode 100 according to an embodiment of the present invention will be described with reference to FIG. 1A, FIG. 1B, FIG. 1C, and FIG. 1D. FIG. 1C illustrates a cross section taken along a line aa′ of FIG. 1B. Further, FIG. 1D illustrates a cross section taken along a line bb′ of FIG. 1B.

The bioelectrode 100 includes an annular sheet 101 that is annular in a plan view, an annular metal-made wiring 102 formed on the annular sheet 101, a conductive sheet 103 formed on the wiring 102, a connection wiring 104, and an adhesive layer 105 formed so as to cover the conductive sheet 103. The bioelectrode 100 can be attached, via the adhesive layer 105, onto the back surface of a garment.

The annular sheet 101 is configured by a material with insulation, waterproofness, and flexibility, and includes an opening 101a at a central part thereof. The wiring 102 can be configured by, for example, a metal paste. Further, the wiring 102 may be configured by a metal foil.

The conductive sheet 103 is formed on and in contact with the wiring 102, and is electrically connected to the wiring 102. Further, the conductive sheet 103 is formed on the annular sheet 101 (one surface side) so as to cover the wiring 102 and close the opening 101a. Accordingly, on the other surface side of the annular sheet 101, the conductive sheet 103 is exposed at the opening 101a. For example, the conductive sheet 103 is adhesively fixed on the annular sheet 101 on which the wiring 102 is formed by a conductive adhesive or the like. The conductive sheet 103 is, for example, a conductive fabric using fibers on which conductive polymers are coated. The conductive sheet 103 may be configured by a conductive polymer film. The conductive polymer is, for example, PEDOT-PSS [Poly(3,4-ethylenedioxythiophene)-Poly(styrenesulfonate)].

Further, the connection wiring 104 is connected to the wiring 102 and is drawn out from an annular part of the annular sheet 101 to the outside. Using the connection wiring 104, the wiring 102 is electrically connected to a measuring device. For example, the annular sheet 101 includes a wiring holding portion 101b that protrudes from the annular part to the outside, and the connection wiring 104 is formed on the wiring holding portion 101b. Further, a waterproof film 106 with waterproofness covers the connection wiring 104.

Here, the adhesive layer 105 can be configured by a material with waterproofness. Configuring the adhesive layer 105 by the material with waterproofness can prevent moisture from permeating (infiltrating) into the conductive sheet 103 from the adhesive layer 105 side. Further, although not illustrated, a configuration that a waterproof sheet with waterproofness is provided in the entire area between the conductive sheet 103 and the adhesive layer 105 may be adopted. Including such a waterproof sheet can prevent moisture from permeating (infiltrating) into the conductive sheet 103 from the adhesive layer 105.

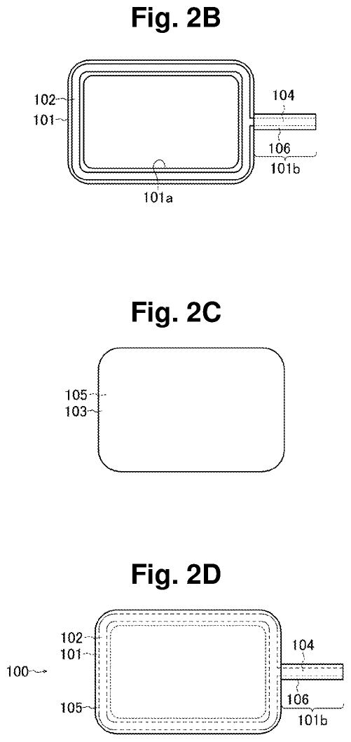

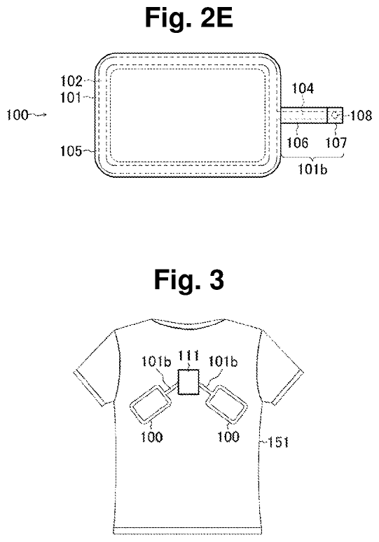

Next, fabrication of the bioelectrode 100 according to an embodiment will be described with reference to FIG. 2A to FIG. 2E. First, as illustrated in FIG. 2A, the wiring 102 and the connection wiring 104 are formed on the annular sheet 101. For example, the wiring 102 and the connection wiring 104 can be fabricated by forming a pattern of the metal paste, such as silver paste, by a screen printing method or the like.

Next, as illustrated in FIG. 2B, the waterproof film 106 is formed on the connection wiring 104 in the wiring holding portion 101b. For example, attaching a polymer material film with waterproofness can obtain the waterproof film 106.

Next, as illustrated in FIG. 2C, the conductive sheet 103 is prepared, and the adhesive layer 105 is attached onto one surface of the prepared conductive sheet 103.

Next, the conductive sheet 103 having the adhesive layer 105 attached on one surface thereof is attached onto the surface of the annular sheet 101 on which the wiring 102 is formed to obtain the bioelectrode 100, as illustrated in FIG. 2D. Subsequently, as illustrated in FIG. 2E, a terminal 107 is attached in a distal end region of the wiring holding portion 101b. The terminal 107 is electrically connected to the connection wiring 104 via a through hole 108 formed in the waterproof film 106.

Next, an application example of the bioelectrode 100 will be described with reference to FIG. 3. The bioelectrode 100 can be attached onto the back surface of a shirt 151 when it is used. Two bioelectrodes 100 are attached onto the back surface of the shirt 151 so that the position of the heart is interposed between these bioelectrodes. Each of the two bioelectrodes 100 is connected to a measuring device 11 via a connection wiring (not illustrated) provided in the wiring holding portion 101b. The measuring device 11 is, for example, an electrocardiographic measuring device, which has a wireless communication function. The measuring device 11 can measure the electrocardiogram from the electric potential difference occurring at the time of muscle contraction of the heart, which is measured by the two bioelectrodes 100. Further, the wireless communication function of the measuring device 111 can be used to transmit the measured electrocardiogram or cardiac rate to a terminal device such as a smartphone.

When a user wears the shirt 151, the conductive sheet 103 of the bioelectrode 100 attached on the back surface of the shirt 151 is brought into a state where it is exposed from the opening 101a of the annular sheet 101 and is in contact with a user's body surface. The conductive sheets 103 of the two bioelectrodes 100 come into contact with the user's body surface so that the position of the heart is interposed between the conductive sheets. On the other hand, the wiring 102 (the connection wiring 104) does not come into contact with the user's body surface because there is the annular sheet 101 intervening between the wiring 102 and the user's body surface.

The electric potential occurring at the time of muscle contraction of the heart is conducted via a path consisting of the conductive sheet 103 being in contact with the body surface, the wiring 102, and the connection wiring 104, and is measured by the measuring device 11. In this manner, the measuring device 11 measures the electrocardiogram from the electric potential difference occurring at the time of muscle contraction of the heart measured by the two bioelectrodes 100.

According to the bioelectrode 100 of the embodiment, the wiring 102 is connected to the conductive sheet 103 that can be brought into contact with the body surface, and the wiring 102 is connected to the measuring device 111 via the connection wiring 104. Accordingly, as compared with the conventional example in which only the conductive sheet is used to form the bioelectrode, the electric resistance between the conductive sheet 103 and the measuring device 11 is lower and higher conductivity can be obtained. As a result, user's biological information such as user's electrocardiogram can be measured more accurately.

Further, the bioelectrode 100 is attached to the shirt 151 via the adhesive layer 105 configured by the material with waterproofness. Accordingly, the insulation isolation between the bioelectrode 100 and the shirt 151 can be secured. Therefore, even if the shirt 151 gets wet with sweat when the user wearing the shirt 151 sweats, and the electric resistance decreases, the insulation isolation between two bioelectrodes 100 can be secured, and the electrocardiogram can be accurately measured.

According to the bioelectrode 100 of the above described embodiment, the conductivity of the bioelectrode using conductive polymers can be further increased.

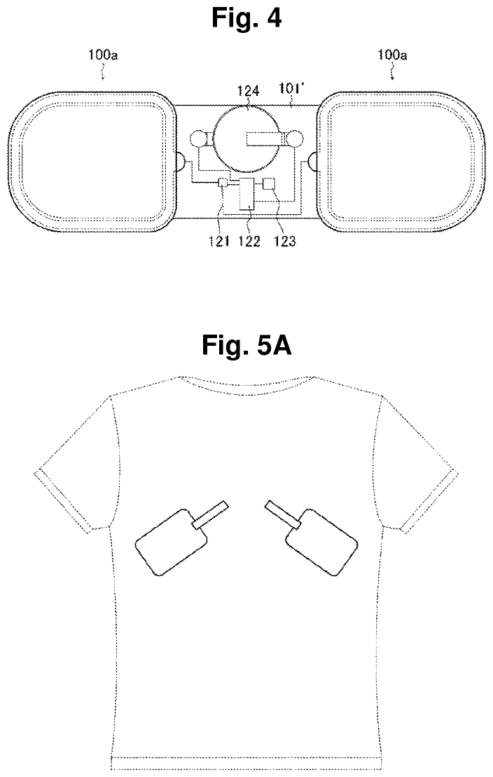

As illustrated in FIG. 4, mounting a measuring device 122 incorporating an electric potential measuring circuit on a sheet 101′ with insulation, waterproofness, and flexibility can configure a system for measuring the electrocardiogram, myoelectricity, and surface potential. The sheet 101′ mounts, in a central part thereof, an A/D converter 121, the measuring device 122, a wireless communication circuit 123, a battery 124, and the like. Further, the sheet 101′ includes two bioelectrodes 100a positioned on both sides of the central part. The sheet 101′ integrates two annular sheets 101. The electric potentials measured by the two bioelectrodes 100a are digitally converted by the A/D converter 121 and measured by the measuring device 122. The measuring device 122 measures the electrocardiogram from the difference between the two digitally converted values. Further, the electrocardiogram measured by the measuring device 122 is transmitted to a wireless terminal by the wireless communication circuit 123.

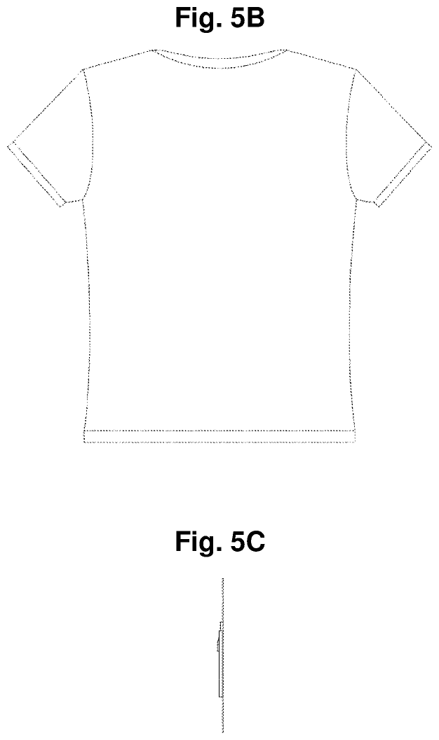

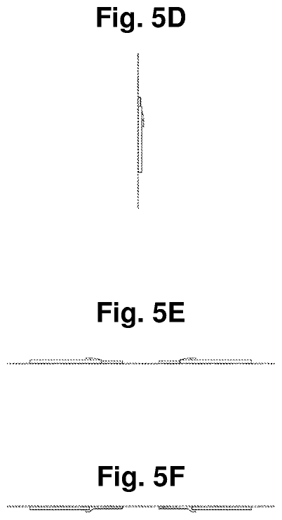

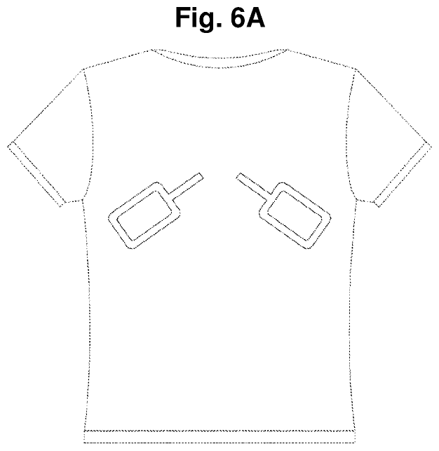

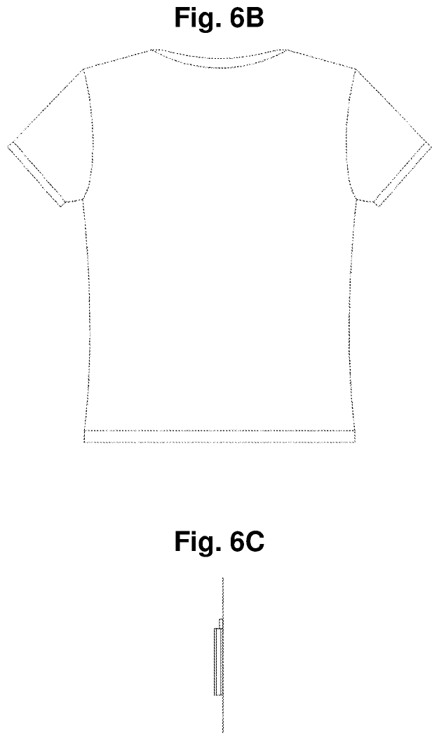

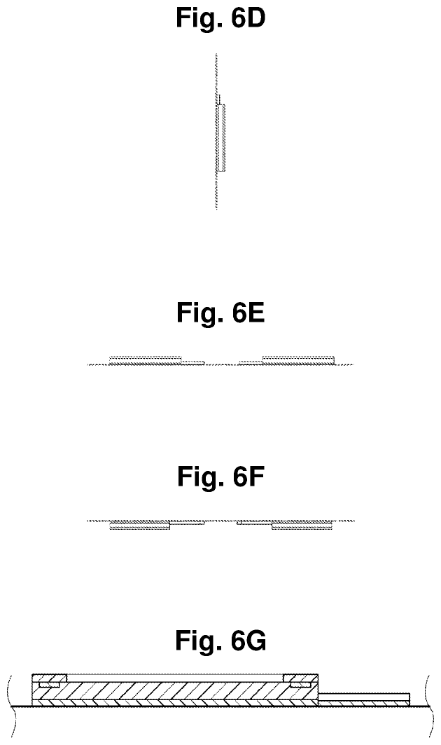

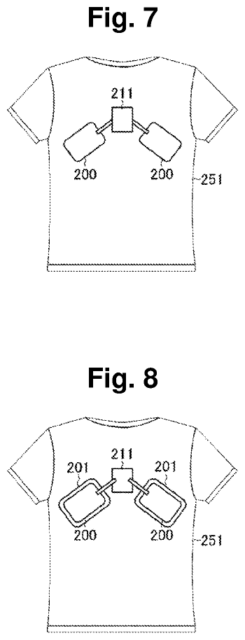

Hereinafter, detailed configurations of bioelectrodes are illustrated in FIG. 5A, FIG. 5B, FIG. 5C, FIG. 5D, FIG. 5E, and FIG. 5F. In addition, detailed configurations of the bioelectrodes according to embodiments of the present invention are illustrated in FIG. 6A, FIG. 6B, FIG. 6C, FIG. 6D, FIG. 6E, FIG. 6F, and FIG. 6G.

As described above, according to embodiments of the present invention, the annular metal-made wiring is provided on the annular sheet that is annular and has the opening at the central part thereof, and the conductive sheet is formed on the annular sheet so as to cover the wiring and close the opening. Therefore, the conductivity of the bioelectrode using conductive polymers can be further increased. Further, according to embodiments of the present invention, the annular sheet is made of the material with waterproofness, and the adhesive layer is made of the material with waterproofness. Further, the waterproof sheet with waterproofness is provided in the entire area between the conductive sheet and the adhesive layer. Therefore, the conductivity can be improved and higher waterproofness can be obtained.

The present invention is not limited to the above described embodiment, and it is apparent that many modifications and combinations can be carried out by those who have ordinary knowledge in the art within technical ideas of the present invention.

### REFERENCE SIGNS LIST

100 bioelectrode
101 annular sheet
101a opening
101b wiring holding portion
102 wiring
103 conductive sheet
104 connection wiring
105 adhesive layer
106 waterproof film.

1.-4. (canceled)

5. A bioelectrode comprising:
an annular sheet with insulation, waterproofness, and flexibility, the annular sheet comprising an opening in a central part thereof;
an annular metal-comprising wiring on the annular sheet;
a conductive sheet on the annular sheet, the conductive sheet covering the annular metal-comprising wiring and closing the opening, the conductive sheet comprising one or more conductive polymers;
a connection wiring connected to the annular metal-comprising wiring and drawn out from an annular part of the annular sheet to outside of the annular sheet; and
an adhesive layer on the conductive sheet, the adhesive layer covering the conductive sheet.

6. The bioelectrode according to claim 5, wherein the adhesive layer has waterproofness.

7. The bioelectrode according to claim 5, further comprising a waterproof sheet with waterproofness, the waterproof sheet spanning an entire area between the conductive sheet and the adhesive layer.

8. The bioelectrode according to claim 5, further comprising a waterproof film with waterproofness, the waterproof film covering the connection wiring.

9. A method of forming a bioelectrode, the method comprising:
providing an annular sheet with insulation, waterproofness, and flexibility, the annular sheet comprising an opening in a central part thereof;
forming an annular metal-comprising wiring on the annular sheet;
forming a conductive sheet on the annular sheet such that the conductive sheet covers the annular metal-comprising wiring and closing the opening, the conductive sheet comprising one or more conductive polymers;
forming a connection wiring connected to the annular metal-comprising wiring and extending from the annular sheet to outside of the annular sheet; and
forming an adhesive layer on the conductive sheet, the adhesive layer covering the conductive sheet.

10. The method of forming the bioelectrode according to claim 9, wherein the adhesive layer is waterproof.

11. The method of forming the bioelectrode according to claim 9, further comprising forming a waterproof sheet with waterproofness, the waterproof sheet spanning an entire area between the conductive sheet and the adhesive layer.

12. The method of forming the bioelectrode according to claim 9, further comprising forming a waterproof film with waterproofness, the waterproof film covering the connection wiring.
